(19) United States
(12) Patent Application Publication
(10) Pub. No.: US 2010/0009017 A1
(43) Pub. Date: Jan. 14, 2010

(54) Anticancer Methods Using Extracts of Anemarrhena asphodeloides Bunge

(75) Inventor: Isaac Cohen, Piedmont, CA (US)

Correspondence Address: WILSON, SONSINI, GOODRICH & ROSATI, 650 PAGE MILL ROAD, PALO ALTO, CA, 94304-1050, US

(73) Assignee: BIONOVO, INC., Emeryville, CA (US)

(21) Appl. No.: 12/422,124

(22) Filed: Apr. 10, 2009

Related U.S. Application Data
(60) Provisional application No. 61/044,403, filed on Apr. 11, 2008.
(60) Provisional application No. 61/094,005, filed on Sep. 3, 2008.

(51) Int. Cl. A61K 36/00; A61K 31/7048; A61P 35/00

(52) U.S. Cl. 424/725; 514/26

(57) ABSTRACT

Selective apoptotic extracts of Anemarrhena asphodeloides Bunge are provided. Also provided are methods of using said extracts to induce apoptosis in specific cells, especially in a human. Provided as well are uses of the extracts of Anemarrhena asphodeloides Bunge for the preparation of a medicament for the selective induction of apoptosis in a living being.

### PRIORITY

The application claims priority under 35 U.S.C. § 119(3) to U.S. provisional patent application 61/044,403, filed Apr. 11, 2008, and from U.S. provisional patent application, 61/094,005, filed Sep. 3, 2008, each of which is incorporated herein by reference in its entirety.

### FIELD OF THE INVENTION

The present invention relates to plant extract compositions, and more particularly to compositions comprising extracts of plant species belonging to the species Anemarrhena asphodeloides Bunge. The invention further relates to methods of using and methods of making such plant extract compositions.

### BACKGROUND

A hallmark feature of cancerous cells is uncontrolled proliferation. Among the causes of uncontrolled proliferation that have been identified, an apparently important one is resistance to the process of programmed cell death, also known as apoptosis. Apoptosis is a process multicellular organisms employ to prevent uncontrolled cell proliferation and to eliminate cells that have become sick, malignant, or superfluous. The process of apoptosis involves a multi-step cascade in which cells are degraded from within through the concerted action of proteolytic enzymes and DNA endonucleases, resulting in the formation of apoptotic bodies that are then removed by scavenger cells. Research to date has shown that much of the intracellular degradation is carried out through the action of the caspases, a family of proteolytic enzymes that cleave adjacent to aspartate residues.

Despite recent advances in breast cancer treatments, current treatment regimes often lead to toxic (sometimes treatment-limiting) side effects. Moreover, current treatments are mostly ineffective against metastatic breast cancer. While early screening and treatment can improve prognosis for many patients, such screening is not uniform and some cancers propagate too quickly to be detected in an early stage by routine screening. There remains a need for treatment options that are less toxic, active against later-stage cancers or both.

Various cultivars of Anemarrhena asphodeloides Bunge are grown in the provinces of Hunan, Hubei, Fujian, Jiangsu, Zhejiang and Jiangxi in China. The dried green embryo of the seed is collected in the autumn. It is washed clean and dried.

There is no known report of using extracts of Anemarrhena asphodeloides Bunge for selective apoptotic treatment of cancer in multicellular organisms, such as mammals, and in particular humans.

### SUMMARY OF THE INVENTION

The inventor has determined that compositions comprising an extract of Anemarrhena asphodeloides Bunge have apoptotic effects. In particular compositions comprising Anemarrhena asphodeloides Bunge extracts selectively induce apoptosis in cancerous cells, while non-cancerous cells are resistant to the cytotoxic effects of the extracts. Thus, extracts described herein, i.e. extracts of Anemarrhena asphodeloides Bunge, and compositions comprising such extracts, are selective apoptotic agents useful for the treatment of disease states characterized by hyperproliferation of cells, such as cancer and benign hyperplastic disorders such as BPH and restenosis.

Thus, embodiments described herein provide a method of selectively inducing apoptosis in a multicellular organism, comprising administering to said organism a pharmaceutical composition comprising an amount of an extract of a plant species selected from the taxonomic species Anemarrhena asphodeloides Bunge effective to selectively induce apoptosis in at least one hyperproliferative population of cells. In some embodiments, the organism is a mammalian organism, such as a rat, a mouse, a human, a simian or a dog. In some embodiments, the hyperproliferative population of cells is a cancer. In some embodiments, the cancer is a bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer, uterine cancer. In some embodiments, the hyperproliferative population of cells is a solid tumor. In some embodiments, the solid tumor is breast cancer, uterine cancer, cervical cancer, vaginal cancer or prostate cancer. In some embodiments, the hyperproliferative population of cells is a benign hyperproliferative disease. In some embodiments, the benign hyperproliferative disease is benign prostatic hypertrophy, psoriasis or restenosis. In some embodiments, the hyperproliferative population of cells is metastatic cancer. In some embodiments, the metastatic cancer is metastatic breast cancer.

Some embodiments herein provide for the use of a composition comprising an amount of an extract of amount of an extract of a plant species selected from the taxonomic species Anemarrhena asphodeloides Bunge effective to prepare a medicament capable of selectively inducing apoptosis in at least one hyperproliferative population of cells in a multicellular organism. In some embodiments, the organism is a mammalian organism, such as a rat, a mouse, a human, a simian or a dog. In some embodiments, the hyperproliferative population of cells is a cancer. In some embodiments, the cancer is a bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer, uterine cancer. In some embodiments, the hyperproliferative population of cells is a solid tumor. In some embodiments, the solid tumor is breast cancer, uterine cancer, cervical cancer, vaginal cancer or prostate cancer. In some embodiments, the hyperproliferative population of cells is a benign hyperproliferative disease. In some embodiments, the benign hyperproliferative disease is benign prostatic hypertrophy, psoriasis or restenosis. In some embodiments, the hyperproliferative population of cells is metastatic cancer. In some embodiments, the metastatic cancer is metastatic breast cancer.

Some embodiments described herein provide a method of treating cancer in a multicellular organism, comprising administering to said organism a pharmaceutical composition comprising an amount of an extract of a plant species selected from the taxonomic species Anemarrhena asphodeloides Bunge effective to treat said cancer. In some embodiments, the organism is a mammalian organism, such as a rat, a mouse, a human, a simian or a dog. In some embodiments, the cancer is a bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer, uterine cancer. In some embodiments, the cancer is a solid tumor. In some embodiments, the solid tumor is breast cancer, uterine cancer, cervical cancer, vaginal cancer or prostate cancer. In some embodiments, the cancer is metastatic cancer. In some embodiments, the metastatic cancer is metastatic breast cancer.

Some embodiments described herein provide for the use of a composition comprising an amount of an extract of amount of an extract of a plant species selected from the taxonomic species Anemarrhena asphodeloides Bunge effective for the preparation of a medicament for the treatment of cancer in a multicellular organism. In some embodiments, the organism is a mammalian organism, such as a rat, a mouse, a human, a simian or a dog. In some embodiments, the cancer is a bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer, uterine cancer. In some embodiments, the cancer is a solid tumor. In some embodiments, the solid tumor is breast cancer, uterine cancer, cervical cancer, vaginal cancer or prostate cancer. In some embodiments, the cancer is metastatic cancer. In some embodiments, the metastatic cancer is metastatic breast cancer.

Some embodiments described herein provide a composition for the treatment of cancer in a multicellular organism, comprising an amount of Timosaponin A3 and Timosaponin B2, which is effective to treat cancer in said multicellular organism.

Some embodiments described herein provide a composition for the treatment of cancer in a multicellular organism, comprising an amount of Timosaponin A3, which is effective to treat cancer in said multicellular organism.

Some embodiments described herein provide a composition for the treatment of cancer in a multicellular organism, comprising an amount of Timosaponin B2 effective to treat cancer in said multicellular organism.

Some embodiments described herein provide a method for the treatment of cancer in a multicellular organism, comprising administering to said multicellular organism an effective amount of a pharmaceutical composition comprising Timosaponin A3 and Timosaponin B2.

Some embodiments described herein provide a method for the treatment of cancer in a multicellular organism, comprising administering to said multicellular organism an effective amount of pharmaceutical composition comprising Timosaponin A3.

Some embodiments described herein provide a method for the treatment of cancer in a multicellular organism, comprising administering to said multicellular organism an effective amount of pharmaceutical composition comprising Timosaponin B2.

Some embodiments described herein provide a use of a composition comprising Timosaponin A3, Timosaponin B2 or both Timosaponin A3 and Timosaponin B2, for the manufacture of a medicament for the treatment of cancer in a multicellular organism.

Some embodiments described herein provide a method for selectively inducing apoptosis in a hyperproliferative population of cells in a multicellular organism, comprising administering to said multicellular organism an effective amount of a pharmaceutical composition comprising Timosaponin A3 and Timosaponin B2.

Some embodiments described herein provide a method for selectively inducing apoptosis in a hyperproliferative population of cells in a multicellular organism, comprising administering to said multicellular organism an effective amount of pharmaceutical composition comprising Timosaponin A3.

Some embodiments described herein provide a method for selectively inducing apoptosis in a hyperproliferative population of cells in a multicellular organism, comprising administering to said multicellular organism an effective amount of pharmaceutical composition comprising Timosaponin B2.

Some embodiments described herein provide a use of a composition comprising Timosaponin A3, Timosaponin B2 or both Timosaponin A3 and Timosaponin B2, for the manufacture of a medicament for the selective induction of apoptosis in hyperproliferative cells in a multicellular organism.

Some embodiments described herein provide a composition for the treatment of cancer in a multicellular organism, comprising an amount of an extract of Anemarrhena asphodeloides Bunge, which is effective to treat cancer in said multicellular organism.

Some embodiments described herein provide a method for the treatment of cancer in a multicellular organism, comprising administering to said multicellular organism an effective amount of a pharmaceutical composition comprising an extract of Anemarrhena asphodeloides Bunge.

Some embodiments described herein provide a use of a composition comprising an extract of Anemarrhena asphodeloides Bunge, for the manufacture of a medicament for the treatment of cancer in a multicellular organism.

Some embodiments described herein provide a method for selectively inducing apoptosis in a hyperproliferative population of cells in a multicellular organism, comprising administering to said multicellular organism an effective amount of a pharmaceutical composition comprising an extract of Anemarrhena asphodeloides Bunge.

Some embodiments described herein provide a use of a composition comprising an extract of Anemarrhena asphodeloides Bunge, for the manufacture of a medicament for the selective induction of apoptosis in hyperproliferative cells in a multicellular organism.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: Induction of cell death by BN108. Tumor and non-transformed cell lines and cells were treated with BN108 at 0.5 mg/ml for 24 hours. The chart shows percentage of cells that were binding Annexin V.

FIG. 2. Caspase-4 is activated in BN108-treated cells. BT474 cells were treated with BN108 in presence or absence of specific caspase-4 inhibitor. Activity of caspases-4 and -9 were quantified using Fluorimetric™ assays (Biovision Inc). Apparently, inhibition of caspase 4 leads to the inhibition of caspase-9 activity indicating that caspase-4 activation is apical in the apoptotic process.

FIG. 3. A. BN108 induces REDD1 and inhibits mTORC1 signaling in breast cancer cells. Western blot analysis of expression of REDD1 and phosphorylation of mTOR targets s6 kinase and 4eBP in breast cancer cell line BT474. None of these changes were observed in immortalized non-transformed mammary epithelial cell line MCF10A (not shown). B. BN108 inhibits activity of AKT in breast cancer but not MCF10A cells.

FIG. 4. A. Treatment with BN108 inactivates AKT kinase in breast cancer cells BT474 but not in normal mammary epithelial cells MCF10A. B. SREBP2 is activated by BN108 in both cell lines. Activated AKT was detected using antibody to phosphoserine 473 (Phospho-AKT) that is phosphorylated only in active kinase. Antibody to SREBP detects both inactive precursor of 120 kD and cleaved active nuclear form of 60 kD.

FIG. 5. Effect of Timosaponins A3 and B2 on viability of breast cancer (BT474) and normal epithelial cells (MCF10A). Cells were treated with BN108 at 0.5 mg/ml, Timosaponin A3 at 4 μg/ml, or Timosaponin B2 at 50 μg/ml, inactivated lamarinase alone or lamarinase-treated B2 at 4 μg/ml for 24 hours.

FIG. 6. Pro-apoptotic gene REDD1 is induced in prostate cancer cell line Du145 by treatment with BN108 (0.5 mg/ml) and Timosaponin A3 (7.5 μM).

FIG. 7. Purified Timosaponin A3 and BN108 extract have similar effects on phosphorylation of eIF2a and on activation of SREBP2 as well as expression on SREBP2 target gene IDI1. BT474 cells were treated with TspA3 (5 μM) or BN108 (0.5 mg/ml).

FIG. 8. Purified Timosaponin A3 and BN108 extract have similar effects on cholesterol synthesis on breast cancer cells MM23 and normal MCF10A cells. Cells were treated with either BN108 or TspA3 for indicated times, and cell extracts were analyzed for cholesterol content, which was normalized to the protein content.

FIG. 9. Cytotoxic effects of Timosaponin A3 and BN108 extract are strongly inhibited by cholesterol (CHL, 250 mM) and partially inhibited by caspase 4 inhibitor (LEVD). BT474 cells were treated for 24 hours, and percent of live cells was analyzed on FACScan based on permeability of dead cells for propidium iodide and binding of Annexin V.

FIG. 10. Cytotoxic effect of BN108 in various breast, prostate, immortalized mammary cells, and normal fibroblasts. BN108 caused apoptosis in cancer cells, but relatively little apoptosis was observed in non-cancerous immortalized mammary cells and normal fibroblasts.

FIG. 11. Inhibition of caspase 4 in prostate and breast carcinoma cells treated with BN108 partially protects them from cell death.

FIG. 12. CyQuant cell proliferation assay of BN108 in H460, and A549 cell lines.

FIG. 13. CyQuant cell proliferation assay of BN108 in HCT116, SW480, and DLD1 cell lines.

FIG. 14. CyQuant cell proliferation assay of BN108 in Du145, LNCaP, and PC3 cell lines.

FIG. 15. Conversion of Timosaponin B2 to Timosaponin A3 by lamarinase was confirmed by time of flight mass spectrometry (TOF MS). Disappearance of Timosaponin B2 and appearance of Timosaponin A3 results in an increase in cell death from 16% in the presence of inactivated lamarinase and Timosaponin B2 to 53% in the presence of activated lamarinase and Timosaponin B2.

### DETAILED DESCRIPTION OF THE INVENTION

Disclosed herein are pharmaceutical compositions comprising inter alia an extract of the taxonomic species of plant referred to as Anemarrhena asphodeloides Bunge. Further embodiments disclosed herein provide selectively apoptotic methods of using the herein-described compositions. The selectively apoptotic compositions described herein possess the activity of inducing apoptosis in abnormally dividing cells, such as cancer cells, while not disturbing the normal cellular processes of normal calls. While not desiring to be limited by theory, it is believed that the active ingredients in the disclosed pharmaceutical compositions act through the caspase pathway to induce apoptosis in cells that have otherwise lost their ability to self-regulate through the process of apoptosis. Such active ingredients, which are extracted from Anemarrhena asphodeloides Bunge inhibit the activity of AKT and mTOR kinases in cancer cell, thereby suggesting their activity in inducing or restoring apoptosis in cancerous cells.

Treatment of breast cancer cells with aqueous extract of Anemarrhena asphodeloides Bunge (0.5 mg of the dried, solid extract per mL of aqueous solution) induces significant cell death in many of the cancer cell lines. Normal mammary epithelial cells and fibroblasts are resistant to the cytotoxic effects of the Anemarrhena asphodeloides Bunge extract. Breast cancer cells that were sensitive to the Anemarrhena asphodeloides Bunge extract underwent apoptotic cell death (confirmed by DNA fragmentation, caspase activation, cleavage of PARP and Annexin V staining). In addition to caspase 3, it was observed that activation of caspases 4 and 9, which are linked to apoptosis induced by endoplasmic reticulum stress, was also induced by the 0.5 mg/mL aqueous extract of Anemarrhena asphodeloides Bunge. This solution induced rapid inactivation of AKT and mTOR kinases in breast cancer, but not in non-transformed cells. Expression of several genes that have well-known pro-apoptotic and anti-proliferative characteristics was also induced by the aqueous extract of Anemarrhena asphodeloides Bunge. It is thus an aspect of the invention to take advantage of the selective pro-apoptotic effects of extracts of Anemarrhena asphodeloides Bunge for the treatment of multicellular organisms, such as mammals, and in particular humans.

Some embodiments described herein provide a composition for the treatment of cancer in a multicellular organism, comprising an amount of Timosaponin A3 and Timosaponin B2, which is effective to treat cancer in said multicellular organism. In some embodiments, the composition consists essentially of Timosaponin A3 and Timosaponin B2. In some embodiments, the composition consists of: an amount of Timosaponin A3 and Timosaponin B2 effective to treat cancer in said multicellular organism; and one or more members of the group consisting of excipients, binders, fillers, diluents, capsule formers, slow-release agents, flavorings and taste masking agents. In some embodiments, the composition contains a combined weight of about 50-1,000 mg of Timosaponin A3 and Timosaponin B2, preferably about 100-800 mg of Timosaponin A3 and Timosaponin B2, more preferably about 200-600 mg of Timosaponin A3 and Timosaponin B2. In some embodiments, the combination contains a combined weight of about 1-100 mg of Timosaponin A3 and Timosaponin B2, preferably about 1-50 mg of Timosaponin A3 and Timosaponin B2, more preferably about 1-20 mg of Timosaponin A3 and Timosaponin B2. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a composition for the treatment of cancer in a multicellular organism, comprising an amount of Timosaponin A3, which is effective to treat cancer in said multicellular organism. In some embodiments, the composition consists essentially of an amount of Timosaponin A3, which is effective to treat cancer in said multicellular organism. In some embodiments, the composition consists of: an amount of Timosaponin A3 that is effective to treat cancer in said multicellular organism; and one or more members of the group consisting of excipients, binders, fillers, diluents, capsule formers, slow-release agents, flavorings and taste masking agents. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a composition for the treatment of cancer in a multicellular organism, comprising an amount of Timosaponin B2 effective to treat cancer in said multicellular organism. In some embodiments, the composition consists essentially of an amount of Timosaponin B2, which is effective to treat cancer in said multicellular organism. In some embodiments, the composition consists of: an amount of Timosaponin B2, which is effective to treat cancer in said multicellular organism; and one or more members of the group consisting of excipients, binders, fillers, diluents, capsule formers, slow-release agents, flavorings and taste masking agents. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a method for the treatment of cancer in a multicellular organism, comprising administering to said multicellular organism an effective amount of a pharmaceutical composition comprising Timosaponin A3 and Timosaponin B2. In some embodiments, the pharmaceutical composition consists essentially of an amount of Timosaponin A3 and Timosaponin B2 effective to treat cancer in said multicellular organism. In some embodiments, the pharmaceutical composition consists of: an amount of Timosaponin A3 and Timosaponin B2 effective to treat cancer in said multicellular organism; and one or more members of the group consisting of excipients, binders, fillers, diluents, capsule formers, slow-release agents, flavorings and taste masking agents. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a method for the treatment of cancer in a multicellular organism, comprising administering to said multicellular organism an effective amount of pharmaceutical composition comprising Timosaponin A3. In some embodiments, the pharmaceutical composition consists essentially of an amount of Timosaponin A3 effective to treat cancer in said multicellular organism. In some embodiments, the pharmaceutical composition consists of: an amount of Timosaponin A3 effective to treat cancer in said multicellular organism; and one or more members of the group consisting of excipients, binders, fillers, diluents, capsule formers, slow-release agents, flavorings and taste masking agents. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a method for the treatment of cancer in a multicellular organism, comprising administering to said multicellular organism an effective amount of pharmaceutical composition comprising Timosaponin B2. In some embodiments, the pharmaceutical composition consists essentially of an amount of Timosaponin B2, which is effective to treat cancer in said multicellular organism. In some embodiments, the pharmaceutical composition consists of: an amount of Timosaponin B2 effective to treat cancer in said multicellular organism; and one or more members of the group consisting of excipients, binders, fillers, diluents, capsule formers, slow-release agents, flavorings and taste masking agents. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a use of a composition comprising Timosaponin A3, Timosaponin B2 or both Timosaponin A3 and Timosaponin B2, for the manufacture of a medicament for the treatment of cancer in a multicellular organism. In some embodiments, the composition consists essentially of an amount of both Timosaponin A3 and Timosaponin B2 effective to treat cancer in said multicellular organism. In some embodiments, the composition consists of: an amount of Timosaponin A3 and Timosaponin B2, which is effective to treat cancer in said multicellular organism; and one or more members of the group consisting of excipients, binders, fillers, diluents, capsule formers, slow-release agents, flavorings and taste masking agents. In some embodiments, the composition contains a combined weight of about 50-1,000 mg of Timosaponin A3 and Timosaponin B2, preferably about 100-800 mg of Timosaponin A3 and Timosaponin B2, more preferably about 200-600 mg of Timosaponin A3 and Timosaponin B2. In some embodiments, the composition contains a combined weight of about 1-100 mg of Timosaponin A3 and Timosaponin B2, preferably about 1-50 mg of Timosaponin A3 and Timosaponin B2, more preferably about 1-20 mg of Timosaponin A3 and Timosaponin B2. In some embodiments, the composition consists essentially of an amount of Timosaponin A3, which is effective to treat cancer in said multicellular organism. In some embodiments, the composition consists of: an amount of Timosaponin A3, which is effective to treat cancer in said multicellular organism; and one or more members of the group consisting of excipients, binders, fillers, diluents, capsule formers, slow-release agents, flavorings and taste masking agents. In some embodiments, the composition contains about 50-1,000 mg of Timosaponin A3, preferably about 100-800 mg of Timosaponin A3, more preferably about 200-600 mg of Timosaponin A3. In some embodiments, the composition contains about 1-100 mg of Timosaponin A3, preferably about 1-50 mg of Timosaponin A3, more preferably about 1-20 mg of Timosaponin A3. In some embodiments, the composition consists essentially of an amount of Timosaponin B2, which is effective to treat cancer in said multicellular organism. In some embodiments, the composition consists of: an amount of Timosaponin B2 that is effective to treat cancer in said multicellular organism; and one or more members of the group consisting of excipients, binders, fillers, diluents, capsule formers, slow-release agents, flavorings and taste masking agents. In some embodiments, the composition contains about 50-1,000 mg of Timosaponin B2, preferably about 100-800 mg of Timosaponin B2, more preferably about 200-600 mg of Timosaponin B2. In some embodiments, the composition contains about 1-100 mg of Timosaponin B2, preferably about 1-50 mg of Timosaponin B2, more preferably about 1-20 mg of Timosaponin B2. In some embodiments, the pharmaceutical composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a method for selectively inducing apoptosis in a hyperproliferative population of cells in a multicellular organism, comprising administering to said multicellular organism an effective amount of a pharmaceutical composition comprising Timosaponin A3 and Timosaponin B2. In some embodiments, the pharmaceutical composition consists essentially of an amount of Timosaponin A3 and Timosaponin B2 effective to selectively induce apoptosis in a hyperproliferative population of cells in said multicellular organism. In some embodiments, the pharmaceutical composition consists of: an amount of Timosaponin A3 and Timosaponin B2 effective to selectively induce apoptosis in a hyperproliferative population of cells in said multicellular organism; and one or more members of the group consisting of excipients, binders, fillers, diluents, capsule formers, slow-release agents, flavorings and taste masking agents. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a method for selectively inducing apoptosis in a hyperproliferative population of cells in a multicellular organism, comprising administering to said multicellular organism an effective amount of pharmaceutical composition comprising Timosaponin A3. In some embodiments, the pharmaceutical composition consists essentially of an amount of Timosaponin A3 effective to selectively induce apoptosis in a hyperproliferative population of cells in said multicellular organism. In some embodiments, the pharmaceutical composition consists of: an amount of Timosaponin A3 effective to selectively induce apoptosis in a hyperproliferative population of cells in said multicellular organism; and one or more members of the group consisting of excipients, binders, fillers, diluents, capsule formers, slow-release agents, flavorings and taste masking agents. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a method for selectively inducing apoptosis in a hyperproliferative population of cells in a multicellular organism, comprising administering to said multicellular organism an effective amount of pharmaceutical composition comprising Timosaponin B2. In some embodiments, the pharmaceutical composition consists essentially of an amount of Timosaponin B2 effective to selectively induce apoptosis in a hyperproliferative population of cells in said multicellular organism. In some embodiments, the pharmaceutical composition consists of: an amount of Timosaponin B2 effective to selectively induce apoptosis in a hyperproliferative population of cells in said multicellular organism; and one or more members of the group consisting of excipients, binders, fillers, diluents, capsule formers, slow-release agents, flavorings and taste masking agents. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a use of a composition comprising Timosaponin A3, Timosaponin B2 or both Timosaponin A3 and Timosaponin B2, for the manufacture of a medicament for the selective induction of apoptosis in hyperproliferative cells in a multicellular organism. In some embodiments, the composition consists essentially of an amount of both Timosaponin A3 and Timosaponin B2 effective to a induce apoptosis in a hyperproliferative population of cells in said multicellular organism. In some embodiments, the composition consists of: an amount of Timosaponin A3 and Timosaponin B2, which is effective to a induce apoptosis in a hyperproliferative population of cells in said multicellular organism; and one or more members of the group consisting of excipients, binders, fillers, diluents, capsule formers, slow-release agents, flavorings and taste masking agents. In some embodiments, the composition contains a combined weight of about 50-1,000 mg of Timosaponin A3 and Timosaponin B2, preferably about 100-800 mg of Timosaponin A3 and Timosaponin B2, more preferably about 200-600 mg of Timosaponin A3 and Timosaponin B2. In some embodiments, the composition contains a combined weight of about 1-100 mg of Timosaponin A3 and Timosaponin B2, preferably about 1-50 mg of Timosaponin A3 and Timosaponin B2, more preferably about 1-20 mg of Timosaponin A3 and Timosaponin B2. In some embodiments, the composition consists essentially of an amount of Timosaponin A3, which is effective to a induce apoptosis in a hyperproliferative population of cells in said multicellular organism. In some embodiments, the composition consists of: an amount of Timosaponin A3, which is effective to a induce apoptosis in a hyperproliferative population of cells in said multicellular organism; and one or more members of the group consisting of excipients, binders, fillers, diluents, capsule formers, slow-release agents, flavorings and taste masking agents. In some embodiments, the composition contains about 50-1,000 mg of Timosaponin A3, preferably about 100-800 mg of Timosaponin A3, more preferably about 200-600 mg of Timosaponin A3. In some embodiments, the composition contains about 1-100 mg of Timosaponin A3, preferably about 1-50 mg of Timosaponin A3, more preferably about 1-20 mg of Timosaponin A3. In some embodiments, the composition consists essentially of an amount of Timosaponin B2, which is effective to a induce apoptosis in a hyperproliferative population of cells in said multicellular organism. In some embodiments, the composition consists of: an amount of Timosaponin B2 that is effective to a induce apoptosis in a hyperproliferative population of cells in said multicellular organism; and one or more members of the group consisting of excipients, binders, fillers, diluents, capsule formers, slow-release agents, flavorings and taste masking agents. In some embodiments, the composition contains about 50-1,000 mg of Timosaponin B2, preferably about 100-800 mg of Timosaponin B2, more preferably about 200-600 mg of Timosaponin B2. In some embodiments, the composition contains about 1-100 mg of Timosaponin B2, preferably about 1-50 mg of Timosaponin B2, more preferably about 1-20 mg of Timosaponin B2. In some embodiments, the pharmaceutical composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a composition for the treatment of cancer in a multicellular organism, comprising an amount of an extract of Anemarrhena asphodeloides Bunge, which is effective to treat cancer in said multicellular organism. In some embodiments, the composition consists essentially of an extract of Anemarrhena asphodeloides Bunge. In some embodiments, the composition consists of: an amount of an extract of Anemarrhena asphodeloides Bunge effective to treat cancer in said multicellular organism; and one or more members of the group consisting of excipients, binders, fillers, diluents, capsule formers, slow-release agents, flavorings and taste masking agents. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments set forth herein provide a method for the treatment of cancer in a multicellular organism, comprising administering to said multicellular organism an effective amount of a pharmaceutical composition comprising an extract of Anemarrhena asphodeloides Bunge. In some embodiments, the pharmaceutical composition consists essentially of an amount of an extract of Anemarrhena asphodeloides Bunge effective to treat cancer in said multicellular organism. In some embodiments, the pharmaceutical composition consists of: an amount of an extract of Anemarrhena asphodeloides Bunge effective to treat cancer in said multicellular organism; and one or more members of the group consisting of excipients, binders, fillers, diluents, capsule formers, slow-release agents, flavorings and taste masking agents. In some embodiments, the pharmaceutical composition consists of: an effective amount of an extract of Anemarrhena asphodeloides Bunge; and one or more members of the group consisting of excipients, binders, fillers, diluents, capsule formers, slow-release agents, flavorings and taste masking agents. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments set forth herein provide a use of a composition comprising an extract of Anemarrhena asphodeloides Bunge, for the manufacture of a medicament for the treatment of cancer in a multicellular organism. In some embodiments, the composition consists essentially of an amount of an extract of Anemarrhena asphodeloides Bunge effective to treat cancer in said multicellular organism. In some embodiments, the composition consists of: an amount of an extract of Anemarrhena asphodeloides Bunge, which is effective to treat cancer in said multicellular organism; and one or more members of the group consisting of excipients, binders, fillers, diluents, capsule formers, slow-release agents, flavorings and taste masking agents. In some embodiments, the composition contains a combined weight of about 50-50,000 mg of an extract of Anemarrhena asphodeloides Bunge, preferably about 1000-40,000 mg of an extract of Anemarrhena asphodeloides Bunge, more preferably about 2000-35,000 mg of an extract of Anemarrhena asphodeloides Bunge. In some embodiments, the composition contains a combined weight of about 100-1000 mg of an extract of Anemarrhena asphodeloides Bunge, preferably about 100-500 mg of an extract of Anemarrhena asphodeloides Bunge, more preferably about 10-200 mg of an extract of Anemarrhena asphodeloides Bunge. In some embodiments, the pharmaceutical composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments described herein provide a method for selectively inducing apoptosis in a hyperproliferative population of cells in a multicellular organism, comprising administering to said multicellular organism an effective amount of a pharmaceutical composition comprising an extract of Anemarrhena asphodeloides Bunge. In some embodiments, the pharmaceutical composition consists essentially of an amount of an extract of Anemarrhena asphodeloides Bunge effective to selectively induce apoptosis in a hyperproliferative population of cells in said multicellular organism. In some embodiments, the pharmaceutical composition consists of: an amount of an extract of Anemarrhena asphodeloides Bunge effective to selectively induce apoptosis in a hyperproliferative population of cells in said multicellular organism; and one or more members of the group consisting of excipients, binders, fillers, diluents, capsule formers, slow-release agents, flavorings and taste masking agents. In some embodiments, the composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

Some embodiments set forth herein provide use of a composition comprising an extract of Anemarrhena asphodeloides Bunge, for the manufacture of a medicament for the selective induction of apoptosis in hyperproliferative cells in a multicellular organism. In some embodiments, the composition consists essentially of an amount of an extract of Anemarrhena asphodeloides Bunge effective to a induce apoptosis in a hyperproliferative population of cells in said multicellular organism. In some embodiments, the composition consists of: an amount of an extract of Anemarrhena asphodeloides Bunge, which is effective to a induce apoptosis in a hyperproliferative population of cells in said multicellular organism; and one or more members of the group consisting of excipients, binders, fillers, diluents, capsule formers, slow-release agents, flavorings and taste masking agents. In some embodiments, the composition contains a combined weight of about 50-50,000 mg of an extract of Anemarrhena asphodeloides Bunge, preferably about 1000-40,000 mg of an extract of Anemarrhena asphodeloides Bunge, more preferably about 2000-35,000 mg of an extract of Anemarrhena asphodeloides Bunge. In some embodiments, the composition contains a combined weight of about 100-1000 mg of an extract of Anemarrhena asphodeloides Bunge, preferably about 100-500 mg of an extract of Anemarrhena asphodeloides Bunge, more preferably about 10-200 mg of an extract of Anemarrhena asphodeloides Bunge. In some embodiments, the pharmaceutical composition is in an oral dosage form. In some embodiments, the multicellular organism is a rat, a mouse, a human, a simian or a dog. In some embodiments, the multicellular organism is a human. In some embodiments, the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

As used herein, the terms “comprising”, “comprises”, “comprise” and grammatical variants thereof are inclusive or open-ended and do not exclude additional, unrecited elements or method steps. The terms “include”, “includes”, “contain”, “contains”, “containing” and grammatical variants thereof are likewise inclusive.

As used herein, the phrase “consisting of” excludes any element, step, or ingredient not specified in the following portion of the sentence.

As used herein, the phrase “consisting essentially of” limits the scope of the following part of the sentence to the specified materials or steps and those that do not materially affect the basic and novel characteristic(s) of the claimed invention.

### Extracts of Anemarrhena Asphodeloides Bunge

In some embodiments, the plant species are of the plant species Anemarrhena asphodeloides Bunge are various cultivars of Anemarrhena asphodeloides Bunge.

Plant matter means any part or parts of at least one plant from the species Anemarrhena asphodeloides Bunge. Plant matter includes the whole plant or any part or parts of the plant, such as the root, bark, wood, leaves, flowers (or flower such as: sepals, petals, stamens, pistils, etc.), fruit, seeds and/or parts or mixtures of any of the foregoing. Plant matter may be fresh cut, dried (including freeze dried), frozen, etc. Plant matter may also be whole or separated into smaller parts. For example, leaves may be chopped, shredded or ground; roots may be chopped or ground; fruit may be chopped, sliced or blended; seeds may be chopped or ground; stems may be shredded, chopped or ground. In particular embodiments of the invention, the plant parts used are the rhizomes of Anemarrhena asphodeloides Bunge.

Plant extract compositions of the invention contain at least one extract of an Anemarrhena asphodeloides Bunge. An “extract” is a solution, concentrate or residue that results when a plant part is contacted with an extraction solvent under conditions suitable for one or more compounds from the plant to partition from the plant matter into the extraction solvent; the solution is then optionally reduced to form a concentrate or a residue.

Suitable extraction media for the present invention include water and ethyl alcohol. Specifically, where water is the extraction solvent, purified water is suitable. Purified water includes distilled water, deionized water, water for injection, ultrafiltered water, and other forms purified of water. Ethyl alcohol that is employed in some embodiments of the invention is grain ethanol, and in particular undenatured ethanol (e.g. pure grain ethanol, optionally containing some water, e.g. up to about 10% water). In some embodiments, the extraction solvent is water, ethanol, or a mixture thereof. A concentrate or residue may be prepared by reducing (e.g. evaporating or lyophilizing) the extraction solution. Whether in the original extraction solvent, reduced concentrate, or residue form, each of these preparations is considered an “extract” for the purposes of the invention.

A method of producing the plant extract according to the invention optionally comprises first comminuting the plant matter in order to increase its surface area to volume ratio and to concomitantly increase efficiency of the extraction process. Methods of comminuting plant matter include grinding, chopping, blending, shredding, pulverizing, triturating, etc.

The extraction medium (solvent) is then contacted with the plant matter under conditions suitable for causing one or more phytochemicals, in particular selectively apoptotic phytochemicals, to partition from the plant matter into the extraction medium. Such conditions include, in some cases, heating the extraction medium to a temperature above room temperature, agitation, contact time, etc. Exemplary temperatures for extraction are from about 50° C. to the boiling point of the extraction solvent. Where water is the extraction solvent, the extraction temperature is generally from room temperature to about 100° C.; temperatures of from about 50° C. to about 80° C. are especially suitable, and temperatures of about 75° C. are particularly suitable. In the case of ethanol as an extraction solvent, the extraction temperature is generally from about room temperature to about 78.5° C.; temperatures of from about 50° C. to about 78° C. are especially suitable and a temperature of about 75° C. is particularly suitable. The person of skill in the art will recognize that the proper balance should be drawn between extraction efficiency on the one hand and phytochemical compound stability on the other.

Once the extraction medium and the plant matter are combined, they are optionally agitated to ensure efficient exchange of selectively apoptotic compound from the plant matter into the extraction medium, and are left in contact for a time sufficient to extract a useful amount of phytochemical compound from the plant matter into the extraction medium. After such time has elapsed (e.g. from about 5 min. to about 10 hr., more particularly from about 10 min. to about 5 hr., especially about 30 min. to about 2 hr.), the extraction medium containing the phytochemical compounds is separated from the plant matter. Such separation is accomplished by an art-recognized method, e.g. by filtration, decanting, etc.

A composition according to the invention includes an herein-described plant extract or a composition comprising an herein-described plant extract of the invention. In such embodiments, the herein-described composition will optionally contain one or more additional ingredients. Such additional ingredients may be inert or active. Inert ingredients include solvents, excipients and other carriers. Active ingredients include active pharmaceutical ingredients (APIs), including those that exhibit synergistic activity in combination with the herein-described plant extract.

Some embodiments disclosed herein provide a pharmaceutical compositions comprising an extract of the taxonomic species Anemarrhena asphodeloides Bunge. An “extract” is a composition of matter prepared in part by contacting an extraction medium (solvent) with plant matter under conditions suitable for drawing one or more chemical compounds from the plant matter into the extraction medium, forming an extraction solution. The extraction solution is then separated from the plant matter, and is optionally diluted or concentrated (e.g. by evaporation, sublimation or lyophilization) to form the extract.

The species Anemarrhena asphodeloides Bunge from the Liliaceae Family is also variously referred to as Zhi Mu, and is an evergreen perennial growing to 0.5m by 1 m. It is in flower from August to September. The flowers are hermaphroditic, having both male and female organs. The plant prefers light (sandy), medium (loamy) or heavy (clay) soils, which are acid to neutral in pH and moist. The plant can tolerate strong winds but not maritime exposure.

The extraction medium is a suitable liquid solvent, e.g. ethyl acetate, water, methanol, ethanol or mixtures of two or more thereof. The extraction medium is in some cases ethyl acetate, water, ethanol, methanol or another relatively polar liquid solvent. In some embodiments, the extraction medium is an aqueous alcohol solution, such as an aqueous ethanol or methanol solution. In some embodiments, the extraction medium is an aqueous methanol solution. In some cases, the extraction medium is either diluted or reduced. The extraction medium may be fully reduced, whereby the extract takes the form of a residue (residual extract). Thus, the extract contains at a minimum one or more plant-derived compounds (phytochemicals), optionally dissolved in a solvent, which are drawn into the extraction medium through one or more steps of contacting the extraction medium and the plant or plant parts. A concentrated or residual extract may be reconstituted by adding a suitable diluent, e.g. ethyl acetate, water, methanol and/or ethanol, to form a reconstituted extract.

In some embodiments, compositions comprising plant extracts include pure extracts or partitioned extracts (including extracts in which one or more selectively apoptotic active compounds in the extract have been enriched) and combinations of such extracts with one or more additional ingredients. In some embodiments, the compositions include those in a variety of physical forms, including solid, semi-solid, liquid, colloidal, etc. Where the compositions are intended for pharmaceutical use, the additional ingredients are pharmaceutically acceptable. Where the compositions according to the invention are intended for use in assays or other uses that are not directed toward a living body, the additional ingredient(s) may be either pharmaceutically acceptable or not.

In some embodiments, a pure extract may be combined with one or more organic solvents. Such organic solvents may be of various polarities. In some embodiments, suitable solvents include ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether, and other solvents, including those that are pharmaceutically acceptable and those that are generally regarded as safe (GRAS) for human consumption.

In some embodiments, the compositions comprise pure extracts or combinations of extracts with one or more additional solvents. In some embodiments, the extract includes a partitioned or further purified extract. Partitioning or purification may be conducted using various separation techniques, including chromatography. In some embodiments, the extract is a purified or partitioned extract obtained by means of anion exchange chromatography, cation exchange chromatography, reverse phase chromatography, normal phase chromatography, affinity chromatography or exclusion chromatography, to further concentrate active agents in the extract. In some embodiments, the purified or partitioned extract is obtained via one or more steps of liquid chromatography, such as high performance liquid chromatography (HPLC). In some embodiments, high performance liquid chromatography is preparative scale high performance liquid chromatography. In some embodiments, the HPLC is reverse phase or ion exchange chromatography. Other means of separation may also be used to purify or partition the extract, including separation in a separatory funnel or other bi- or multi-phasic separatory mechanism. In some embodiments, the purified or partitioned extract may be combined with one or more additional active or inactive ingredients, such as solvents, diluents, etc. In some embodiments, suitable solvents may include ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether, and other solvents, including those that are pharmaceutically acceptable and those that are generally regarded as safe (GRAS) for human consumption.

Suitable additional ingredients include solvents. Solvents may be subdivided into pharmaceutically acceptable and non-pharmaceutically acceptable solvents. In this context, it is to be understood that some pharmaceutically acceptable solvents include water for injection (WFI), which may be pH adjusted and/or buffered to a preselected pH or pH range, e.g. from about 2 to about 8, more specifically from about 4.0 to about 7.5, and more particularly from about 4.9 to about 7.2.

Pharmaceutically acceptable solvents may further comprise one or more pharmaceutically acceptable acids, bases, salts or other compounds, such as carriers, excipients, etc. Pharmaceutically acceptable acids include HCl, H₂SO₄H₃PO₄, benzoic acid, etc. Pharmaceutically acceptable bases include NaOH, KOH, NaHCO₃, etc. Pharmaceutically acceptable salts include NaCl, NaBr, KCl, etc. Acids and bases may be added in appropriate proportions to buffer a pharmaceutically acceptable solution at a particular, pre-selected pH, especially a pH in the range of about 2-8, more especially in the range of about 5.0 to about 7.2.

Purification of Timosaponin A3 and Timosaponin B2 from extracts of Anemarrhena Asphodeloides Bunge

The inventor has found that Timosaponin A3 (also referred to herein as “Timosaponin A-III”), which represents approximately 2 mg of every 1000 mg of dried extract of Anemarrhena asphodeloides Bunge, induces of apoptosis in a variety of cancer cells in vitro, but does not induce apoptosis in non-cancerous cells. In addition, the inventor has found that Timosaponin B2 (also referred to herein as “Timosaponin B-II”), which represents approximately 79 mg out of every 1000 mg of dried extract of Anemarrhena asphodeloides Bunge, does not induce apoptosis in vitro. However, Timosaponin B2 may be converted into Timosaponin A3 by lamarinase and could be converted into Timosaponin A3 in vivo through the action of gut flora. Indeed, in vitro experiments have shown that treatment of Timosaponin B2 with lamarinase, an enzyme commonly expressed by gut flora, in vitro renders Timosaponin B2 active to induce apoptosis in cancer cells.

Timosaponin A3 and Timosaponin B2 were separated from an extract of Anemarrhena asphodeloides Bunge by high efficiency liquid chromatography and were identified by time of flight mass spectrometry (TOF-MS). The contribution of Timosaponin A3 and Timosaponin B2 to the total mass of a sample of dried extract of Anemarrhena asphodeloides Bunge were calculated based upon the dry mass of samples of Anemarrhena asphodeloides Bunge and the mass of Timosaponin A3 and Timosaponin B2 recovered at the end of the separation process. The structures and molecular weights of Timosaponin A3 and Timosaponin B2 are given below:

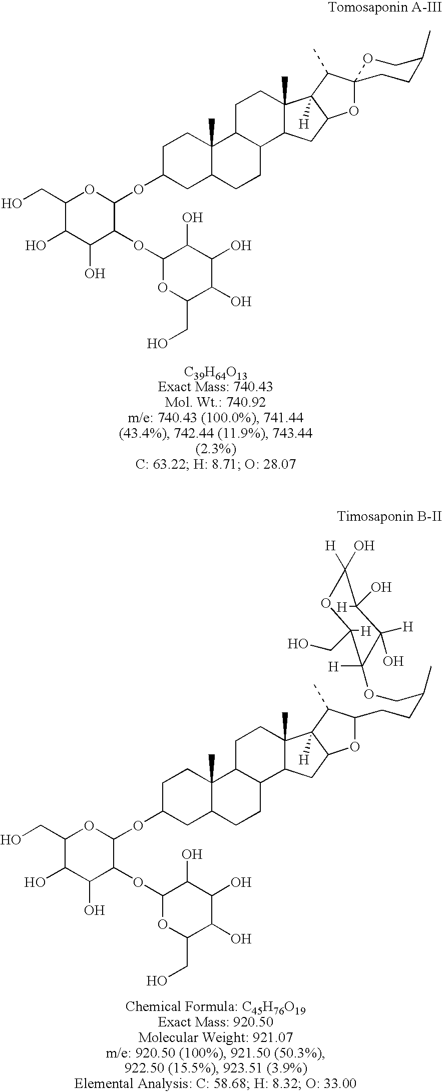

### Pharmaceutical Compositions

Extracts of Anemarrhena asphodeloides Bunge, Timosaponin A3, Timosaponin B2, and mixtures of Timosaponin A3 and Timosaponin B2, may be prepared as above in either solution or dried form. In a solution form, an extract of Anemarrhena asphodeloides Bunge, Timosaponin A3, Timosaponin B2, and mixtures of Timosaponin A3 and Timosaponin B2 may be administered in the form a flavored or unflavored tea. In some embodiments some flavoring, e.g. sweetening, may be desirable to counteract the bitter flavor of the extract. Solutions can also be prepared from dried extract, in tea or elixir forms. Again, flavoring, such as sweetening may be desirable. Taste-masking may be employed to improve patient acceptance of the pharmaceutical composition.

A dried extract of Anemarrhena asphodeloides Bunge, Timosaponin A3, Timosaponin B2, and mixtures of Timosaponin A3 and Timosaponin B2 may be formulated as an orally-available form, such as in a capsule, tablet, caplet, etc. A capsule may be prepared by measuring a suitable amount of the dry extract into one or more gelatin capsule shells and assembling the capsule(s). Tablets and caplets may be prepared by combining the dry extract with one or more excipients, such as pharmaceutically inert binders, fillers, lubricants, diluents, disintegrants, slow-release agents, etc., which are generally known in the art. It is noted that plant fiber is considered a filler for purposes of the present invention. In some embodiments, the invention contemplates solid dosage forms that contain fillers other than plant-derived fiber. Other embodiments contemplate retention of some amount of soluble plant fiber as a filler. Further embodiments contemplate addition of plant fiber as a filler. Tablets, caplets, capsules, etc. may also be coated, e.g. with an enteric coating, to prevent stomach upset or other coating, such as an oxygen-protective barrier.

Either a dried extract of Anemarrhena asphodeloides Bunge, Timosaponin A3, Timosaponin B2, and mixtures of Timosaponin A3 and Timosaponin B2 or a concentrated solution of extract of Anemarrhena asphodeloides Bunge, Timosaponin A3, Timosaponin B2, and mixtures of Timosaponin A3 and Timosaponin B2 may be combined with one or more gelling agents and inserted into a gel capsule. Alternatively, a dried extract of Anemarrhena asphodeloides Bunge, Timosaponin A3, Timosaponin B2, and mixtures of Timosaponin A3 and Timosaponin B2 or a concentrated solution of extract of Anemarrhena asphodeloides Bunge, Timosaponin A3, Timosaponin B2, and mixtures of Timosaponin A3 and Timosaponin B2 may be combined with a gelling agent and optionally one or more flavoring agents for oral administration as an edible gel; or a non-flavored variant may be administered as a rectal suppository gel or gel capsule.

A unit dose of extract of Anemarrhena asphodeloides Bunge is characterized by an equivalent amount of dried extract contained within the dosage form. For example, in some embodiments, a unit dosage may contain 1 mg to about 10 g of dried extract, or the equivalent thereof. In some embodiments, the unit dose will contain about 1 mg to about 10 mg, about 1 mg to about 100 mg, about 1 mg to about 1000 mg (1 g), about 1 mg to about 10000 mg (10 g) of dried extract, or the equivalent thereof. In some embodiments, the unit dose contains about 10 mg to about 100 mg, about 10 mg to about 1000 mg or about 10 mg to about 10000 mg of dried extract or the equivalent thereof. In some embodiments, the unit dose contains about 100 mg to about 5000, about 100 mg to about 2500 mg, about 100 mg to about 2000 mg, about 100 mg to about 1500 mg, about 100 to about 1000, about 100 to about 800 mg of dried extract, or the equivalent thereof. An equivalent of a dried extract of Anemarrhena asphodeloides Bunge is an amount of a dry, liquid, gel or other mixture of Anemarrhena asphodeloides Bunge containing the same amount of apoptotic active as a dried extract of Anemarrhena asphodeloides Bunge. Thus, 30 mL of a tea containing 0.5 mg/mL of dried extract of Anemarrhena asphodeloides Bunge is a unit dose equivalent to 15 mg of dried Anemarrhena asphodeloides Bunge; and a tablet containing 100 mg each of dried extract of Anemarrhena asphodeloides Bunge, a binder, a filler, a disintegrant is equivalent to 100 mg of dried extract neat.

Based upon the observations set forth herein, it is estimated that an effective dose of Timosaponin A3, Timosaponin B2 or a mixture of Timosaponins A3 and B2 would be in the range of about 1-1000 mg/dose, given in single or divided doses, in 1, 2, 3, 4 or more doses per day (approximately 1 to 4000 mg/day). In some embodiments, the dose of Timosaponin A3, Timosaponin B2 or a mixture of Timosaponins A3 and B2 may be about 5 to about 500 mg/dose, given in single or divided doses, in 1, 2, 3, 4 or more doses per day (approximately 5 to 2000 mg/day). In some embodiments, the dose of Timosaponin A3, Timosaponin B2 or a mixture of Timosaponins A3 and B2 may be about 100 mg/dose to about 500 mg/dose, given in single or divided doses, in 1, 2, 3, 4 or more doses per day. In some embodiments, the dose of Timosaponin A3, Timosaponin B2 or a mixture of Timosaponins A3 and B2 may be about 50 mg/dose, about 100 mg/dose, about 150 mg/dose, about 200 mg/dose, about 250 mg/dose, about 300 mg/dose, about 350 mg/dose, about 400 mg/dose, about 450 mg/dose, about 500 mg/dose, about 550 mg/dose, about 600 mg/dose, about 650 mg/dose, about 700 mg/dose, about 750 mg/dose, about 800 mg/dose, given in single or divided doses, in 1, 2, 3, 4 or more doses per day. Thus, in some embodiments, the daily dose of Timosaponin A3, Timosaponin B2 or a mixture of Timosaponins A3 and B2 may be 50 mg/day, about 100 mg/day, about 150 mg/day, about 200 mg/day, about 250 mg/day, about 300 mg/day, about 350 mg/day, about 400 mg/day, about 450 mg/day, about 500 mg/day, about 550 mg/day, about 600 mg/day, about 650 mg/day, about 700 mg/day, about 750 mg/day, about 800 mg/day, about 900 mg/day, about 1000 mg/day, about 1150 mg/day, about 1100 mg/day, about 1200 mg/day, about 1300 mg/day, about 1350 mg/day, about 1400 mg/day, about 1500 mg/day, about 1600 mg/day, about 1650 mg/day, about 1800 mg/day, about 1950 mg/day, about 2000 mg/day, about 2100 mg/day, about 2250 mg/day, about 2400 mg/day, about 2600 mg/day, about 2800 mg/day, about 3000 mg/day, about 3200 mg/day. These amounts of Timosaponin A3, Timosaponin B2, or combination of Timosaponin A3 and Timosaponin B2 may be combined with a suitable liquid, gel or solid excipient, such as a suitable inert carrier, diluent, disintegrant, glidant, binder, flavor, taste-masking agent, slow-release agent to form a suitable dosage form, such as a solution, gel, tablet, caplet or capsule.

### Methods of Treatment

The pharmaceutical compositions comprising extracts of Anemarrhena asphodeloides Bunge and isolated and purified Timosaponin A3, as described herein, possess selective apoptotic activity in cancer cells, such as breast cancer and prostate cancer cells. Moreover Timosaponin B2 is converted to Timosaponin A3 by the activity of lamarinase, and hence would be expected to be converted to Timosaponin A3 in the gut of a mammalian patient, such as a human. Hence, it is expected that pharmaceutical compositions of extracts of Anemarrhena asphodeloides Bunge, as well as pharmaceutical compositions of isolated and purified Timosaponin A3, Timosaponin B2, or combinations of Timosaponin A3 and Timosaponin B2, will have activity in the treatment of various disease states that are characterized by abnormal cell growth, such as that caused by failure of normal apoptotic processes in an organism, organ, tissue or cell line. Among the disease states envisioned as being treatable with the compositions described herein is cancer, including, but not limited to bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer or uterine cancer. In some embodiments the composition described herein is administered to a patient who has been diagnosed with one or more cancers selected from among the solid tumors, such as breast, lung, colon, brain, prostate, stomach, pancreatic, ovarian, skin (melanoma), endocrine, uterine, testicular and bladder cancer.

In some embodiments, pharmaceutical compositions comprising extracts of Anemarrhena asphodeloides Bunge and isolated and purified Timosaponin A3, as described herein, are effective to treat a benign proliferative disease, such as benign prostatic hypertrophy, psoriasis or restenosis (e.g. of an implanted stent).

In some embodiments, pharmaceutical compositions comprising extracts of Anemarrhena asphodeloides Bunge and isolated and purified Timosaponin A3, as described herein, may be combined with another agent that is useful for the treatment of abnormal cell growth, such as cancer, solid tumors, benign hyperproliferative disease, etc. Such additional agent may be selected from among the mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxic agents, anti-hormones, and anti-androgens.

An effective dose of a pharmaceutical compositions comprising extracts of Anemarrhena asphodeloides Bunge and isolated and purified Timosaponin A3, as described herein, is an amount effective to produce a therapeutic effect in a multicellular organism as described herein. In some embodiments, the effective dose is an amount sufficient to induce apoptosis in one or more populations of hyperproliferative cells in the organism. In some embodiments, the effective dose is an amount sufficient to cause relief of one or more symptoms of hyperproliferative cellular disease, such as cancer, in the organism. In some embodiments, the effective dose is an amount sufficient to significantly slow the progression of hyperproliferative cellular disease, to cause partial or complete remission of said hyperproliferative cellular disease, to provide partial or complete prophylaxis against recurrence, spread or malignant growth of said hyperproliferative cellular disease. In some embodiments the dose may be critical to the success of the therapeutic regime. As the extracts of Anemarrhena asphodeloides Bunge are deemed to be largely non-toxic, the effective dose may be varied from about 1 mg to about 10 g per patient per day of dried extract, or the equivalent thereof in a solution or other pharmaceutically acceptable form, as discussed in more detail below. In some embodiments, the effective dose is about 1 mg to about 10 mg, about 1 mg to about 100 mg, about 1 mg to about 1000 mg (1 g), about 1 mg to about 10000 mg (10 g) per patient per day. In some embodiments, the effective dose is about 10 mg to about 100 mg, about 10 mg to about 1000 mg or about 10 mg to about 10000 mg per patient per day. In some embodiments, the effective dose is about 100 mg to about 5000, about 100 mg to about 2500 mg, about 100 mg to about 2000 mg, about 100 mg to about 1500 mg, about 100 to about 1000, about 100 to about 800 mg per patient per day. In some embodiments, treatment days may be altered with non-treatment days. For example, treatment may be commenced on day 1 with an effective dose as described above, with administration of the effective dose repeated on days 3, 5, 7 (or 8), 9, 11, 13, etc. Treatment may be administered once a day for a full week, followed by a week off treatment, followed by at least one additional week on treatment. Treatment with the extract of Anemarrhena asphodeloides Bunge may also be alternated with another anti-cancer treatment, or may be combined with another anti-cancer treatment to take advantage of the combined effects of the cancer treatments.

Additional cancer treatments can include, but are not limited to, surgical excision of all or part of a solid tumor, radiation treatment, adjunctive chemotherapy, anti-inflammatory drugs, analgesic drugs, etc.

Treatment (and its grammatical variants—e.g. treat, to treat, treating, treated, etc.) of a disease, disorder, syndrome, condition or symptom includes those steps that a clinician would take to identify a subject to receive such treatment and to administer a composition of the invention to the subject. Treatment thus includes diagnosis of a disease, syndrome, condition or symptom that is likely to be ameliorated, palliated, improved, eliminated, cured by administering the selectively apoptotic plant extract of the invention to the subject. Treatment also includes the concomitant amelioration, palliation, improvement, elimination, or cure of the disease, disorder, syndrome, condition or symptom. In some embodiments, treatment implies prevention or delay of onset of a disease, disorder, syndrome, condition or symptom (i.e. prophylaxis), prevention or delay of progression of a disease, disorder, syndrome, condition or symptom, and/or reduction in severity of a disease, disorder, syndrome, condition or symptom. In the case of neoplastic growth in particular, treatment includes palliation, as well as the reversal, halting or delaying of neoplastic growth. In this regard, treatment also includes remission, including complete and partial remission. In the case of climacteric symptoms, treatment includes prevention and palliation of various symptoms.

Prevention (and its grammatical variants) of a disease, disorder, syndrome, condition or symptom includes identifying a subject at risk to develop the disease, disorder, syndrome, condition or symptom, and administering to that subject an amount of the herein-described plant extract sufficient to be likely to obviate or delay the onset of said disease, disorder, syndrome, condition or symptom. In some cases, prevention includes identifying a post-menopausal woman who the clinician believes, applying a competent standard of medical care, to be in need of hormone replacement therapy, and administering a plant extract of the present invention to the woman, whereby one or more climacteric symptoms is blocked or delayed. In some embodiments, prevention of osteoporosis includes identifying a post-menopausal woman who the clinician believes, applying a competent standard of medical care, to be at risk for developing osteoporosis, and administering a plant extract of the present invention to the woman, whereby the onset of bone loss is blocked or delayed.

Palliation includes reduction in the severity, number and/or frequency of occurrences of an a disease, disorder, syndrome, condition or symptom. Palliation of climacteric symptoms includes reducing the frequency and/or severity of hot flashes, insomnia, incontinence, depression, etc.

### Administration of Extracts of Anemarrhena Asphodeloides Bunge

Administration of the pharmaceutical compositions according to the present invention will be via a commonly used administrative route so long as one or more of the plant extracts is available to target tissue via that route. Some administrative routes that may be mentioned include: oral, nasal, buccal, rectal, vaginal and/or topical (dermal). Alternatively, administration may be by orthotopic, intradermal, subcutaneous, intramuscular, intraperitoneal or intravenous injection. Such compositions would normally be administered as pharmaceutically acceptable compositions, described supra.

### EXAMPLES

The invention may be more fully appreciated with reference to the following illustrative and non-limiting examples.

### Example 1

### In Vitro Studies

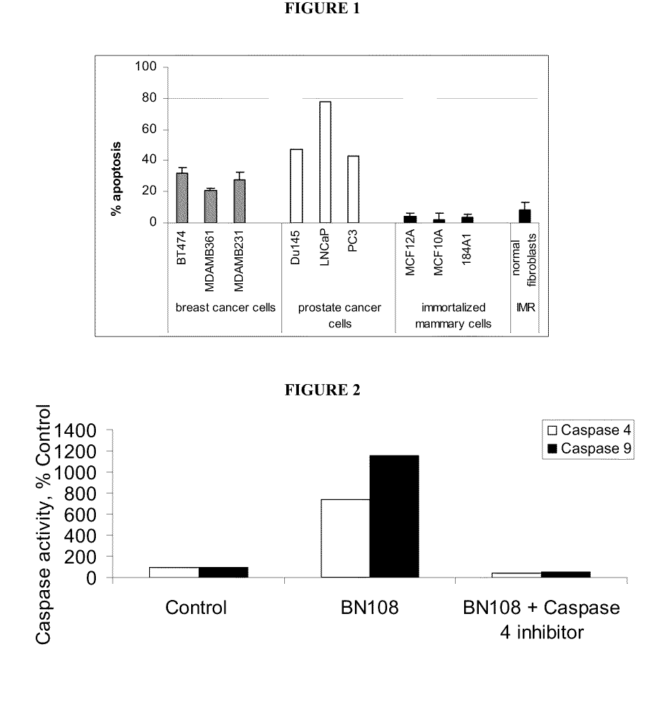

Plant extracts of Anemarrhena asphodeloides Bunge selectively induce apoptosis in cancerous cells. As can be seen in FIG. 1, tumor and non-transformed cell lines and cells were treated with a solution comprising 0.5 mg/mL of dried extract of Anemarrhena asphodeloides Bunge. (The solution containing 0.5 mg/mL of dried extract of Anemarrhena asphodeloides Bunge is also referred to herein as BN108.) FIG. 1 shows the percentage of cells that bound Annexin V after 24 hours of treatment for three breast cancer cell lines, three prostate cancer cell lines, three immortalized mammary cell lines and normal fibroblasts. The percent apoptosis rate for BN108-treated breast and prostate cancer cells ranged from 20% to 80%, while the percent apoptosis rate for BN108-treated immortalized mammary cells was negligible and for BN108-treated normal fibroblasts was less than 10%. Thus, it can be seen that BN108 induced apoptosis in cancer cells but not in normal cells and non-cancerous immortalized cells.

BN108 induces activation of caspase 4 and caspase 9, which are linked to apoptosis induced by endoplasmic reticulum stress, in BT474 cells. As can be seen in FIG. 2, BT474 cells were treated with BN108 in the presence or absence of a specific caspase-4 inhibitor. Activity of caspases-4 and -9 were quantified using fluorometric assays (Biovision, Inc.) Expression levels of both of these apoptosis-linked caspases were increased several fold over their expression levels in the presence of control. The expression levels of both caspase-4 and caspase-9 are reduced below control levels in the presence of BN108 and a caspase-4 inhibitor. Apparently, inhibition of caspase 4 leads to the inhibition of caspase-9 activity, indicating that caspase-4 activation is apical in the apoptotic process induced by BN108.

Treatment of breast cancer cells with BN108 inhibits activity of AKT and mTOR kinases. Expression array analysis of BN108-induced changes identified several transcripts with roles in growth arrest and/or apoptosis that are upregulated. Among the upregulated genes with roles in growth arrest and/or apoptosis are: p21CIP, stratifin, cyclin G2, GDF15 and Bim. A number of mRNAs coding for enzymes in the cholesterol synthesis pathway. Downregulated genes: Growth factors, VEGF, amphiregulin, miR 17-92 and C-myc. Changes in expression of corresponding proteins were confirmed by Western blotting. In addition, BN108 induces expression of REDD1/DDIT4, whose protein product is a negative regulator of mTORC1.

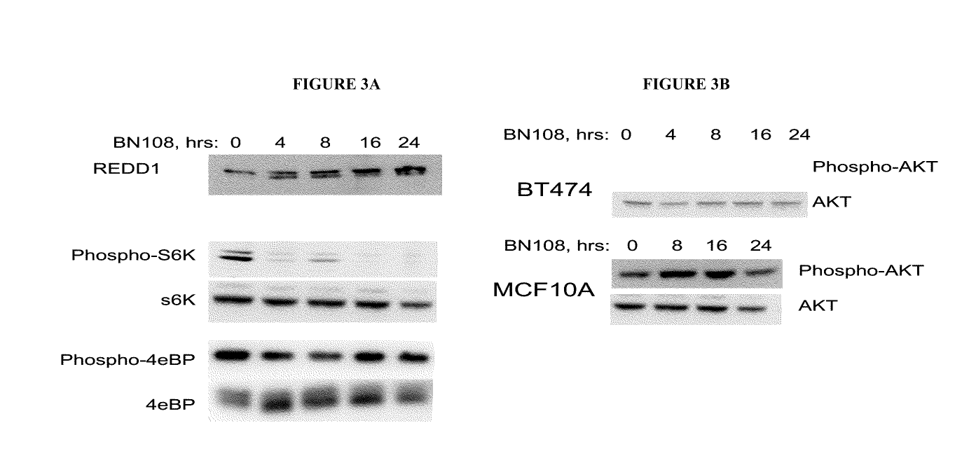

As can be seen in FIG. 3, BN108 induces REDD1, a gene whose expression is associated with apoptosis, and inhibits mTORC1 signaling in breast cancer cells. FIG. 3A shows Western blot analysis of expression of REDD1 and phosphorylation of mTOR targets s6 kinase and 4eBP in breast cancer cell line BT474. None of these changes were observed in immortalized non-transformed mammary epithelial cell line MCF10A (not shown). FIG. 3B shows that BN108 also inhibits activity of AKT in breast cancer but not MCF10A cells.

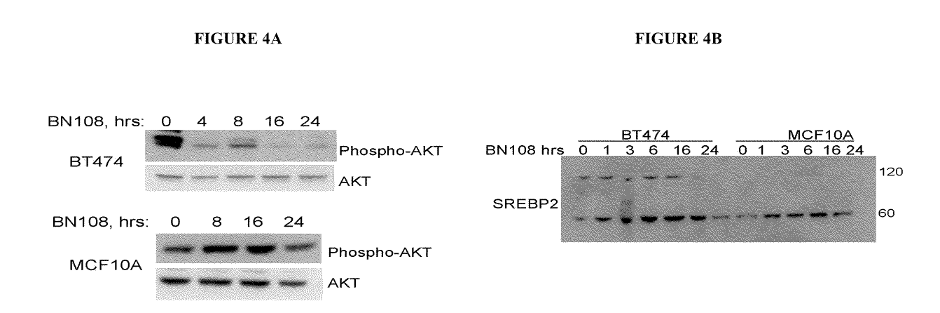

As shown in FIG. 4A, treatment with BN108 inactivates AKT kinase in breast cancers cells BT474, but not in normal mammary epithelial cells MCF10A. In FIG. 4B, it can be seen that SREBP2 is activeated by BN108 in both the cancer cells (BT474) and the non-cancerous cells (MCF10A).

BN108-induced cell death is accompanied by caspase activation and DNA fragmentation. BN108 does not induce generation of reactive oxygen species, DNA damage or mitochondrial dysfunction. BN108 induced rapid inactivation of AKT and mTOR kinases in breast cancer. In particular, induction by BN108 of stress response gene REDD1 could be related to the observed inhibition of activity of mTOR complex, as REDD1 is a negative regulator of TORC.

### Example 2

### Isolation of Timosaponin A3 and Timosaponin B2 from an Extract of Anemarrhena Asphodeloides Bunge

Timosaponin A3 and Timosaponin B2 were separated from an extract of Anemarrhena asphodeloides Bunge as described in general herein above. The isolated Timosaponin A3 and Timosaponin B2 were characterized by GC Mass Spec as described herein above.

### Example 3

### In vitro Experiments with Timosaponin A3 and Timosaponin B2

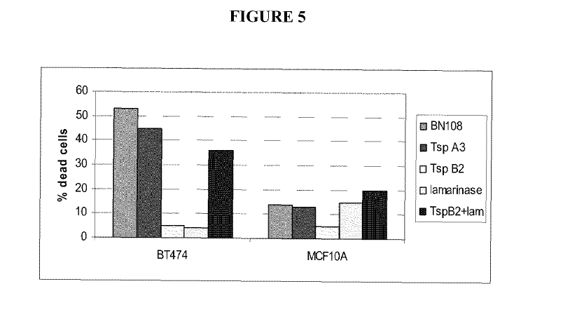

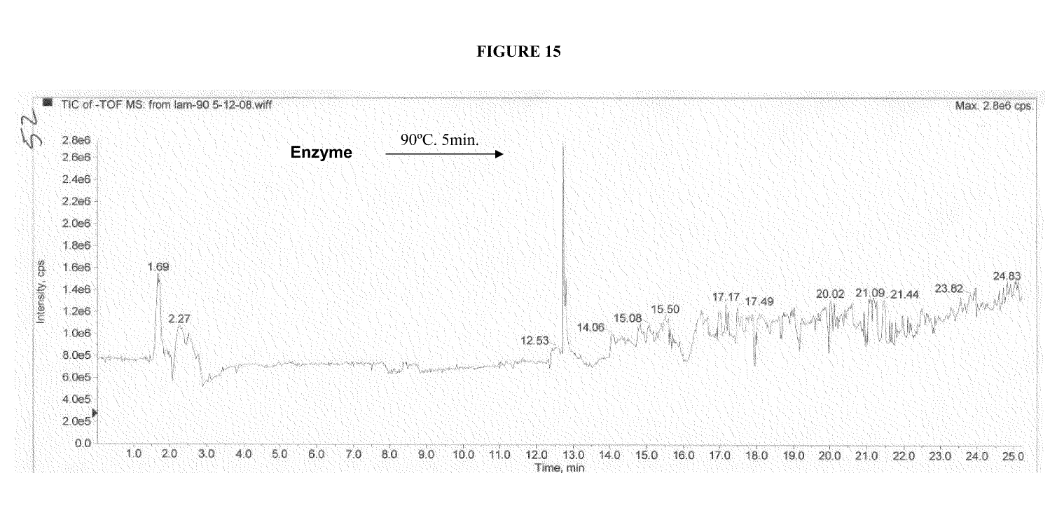

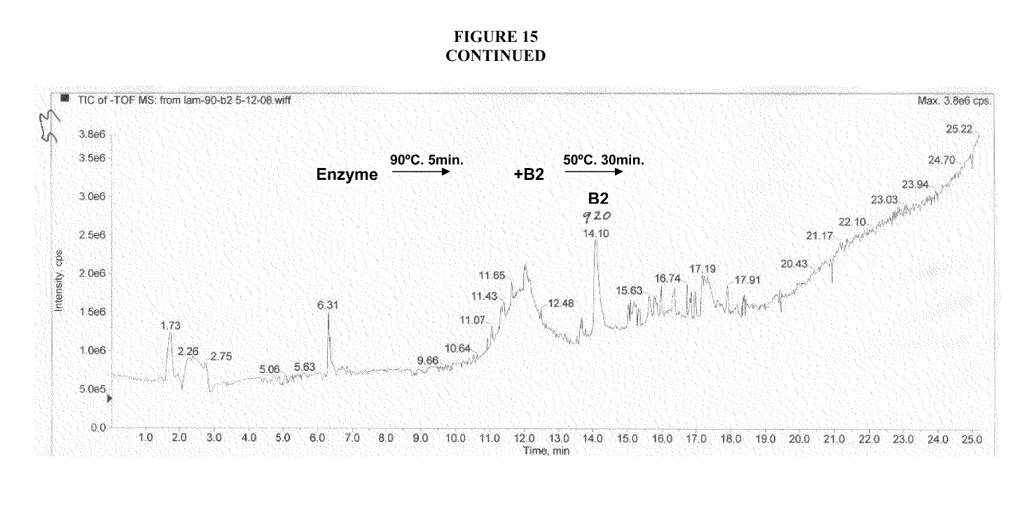

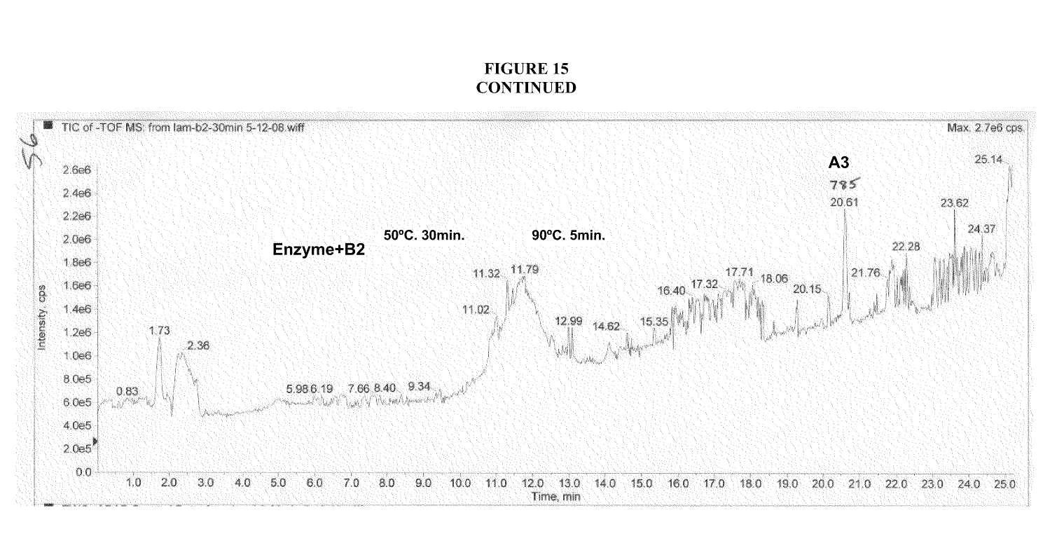

The effect of Timosaponins A3 and B2 on the viability of breast cancer cells and normal epithelial cells was tested by treating cells with either 0.5 mg/mL BN108, 4 μg/mL Timosaponin A3, 50 μg/mL Timosaponin B2, inactivated lamarinase alone or lamarinase-treated Timosaponin B2 (4 μg/mL) for 24 hr. As can be seen in FIG. 5, BN108 and Timosaponin A3 (“Tsp A3”) induced cell death in BT474 cells, whereas Timosaponin B2 (“Tsp B2”) and lamarinase alone did not induce cell death in BT474 cells, and treatment of 4 μg/mL Timosaponin B2 with lamarinase resulted in nearly as great an induction in cell death in BT474 cells as was induced by 4 μg/mL Timosaponin A3. The difference in activity between Timosaponin A3 and lamarinase treated Timosaponin B2 is, on a molar basis, probably negligible, given that the quotient of the molecular weights of Timosaponin B2 (920 Da) and Timosaponin A3 (740 Da) is roughly equivalent to the ratio of the % dead cells in the Timosaponin A3 and the lamarinase-treated Timosaponin B2. Thus, it can be inferred that nearly all the Timosaponin B2 is converted into Timosaponin A3, and is active to induced apoptosis, in the presence of lamarinase. Conversion of Timosaponin B2 to Timosaponin A3 by lamarinase was confirmed by time of flight mass spectrometry (TOF MS), as can be seen in FIG. 15. As can be seen in FIG. 15, disappearance of Timosaponin B2 and appearance of Timosaponin A3 results in an increase in cell death from 16% in the presence of inactivated lamarinase and Timosaponin B2 to 53% in the presence of activated lamarinase and Timosaponin B2.

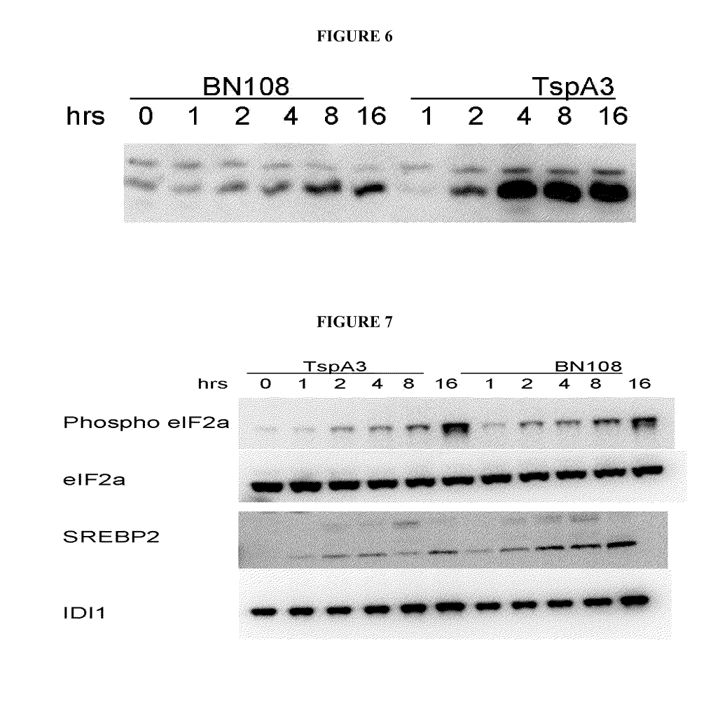

Both BN108 and Timosaponin A3 induce the pro-apoptotic gene REDD1 in prostate cancer cells. As can be seen in FIG. 6, pro-apoptotic gene REDD1 is induced in Du145 prostate cancer cells by treatment with BN108 (0.5 mg/mL dried extract of Anemarrhena asphodeloides Bunge) and Timosaponin A3 (7.5 μM). As can be seen in FIG. 7, Timosaponin A3 (5 μM) and BN108 (0.5 mg/mL) increase phosphorylation of elF2a, increase expression of SREBP2 and increase expression of IDI1.

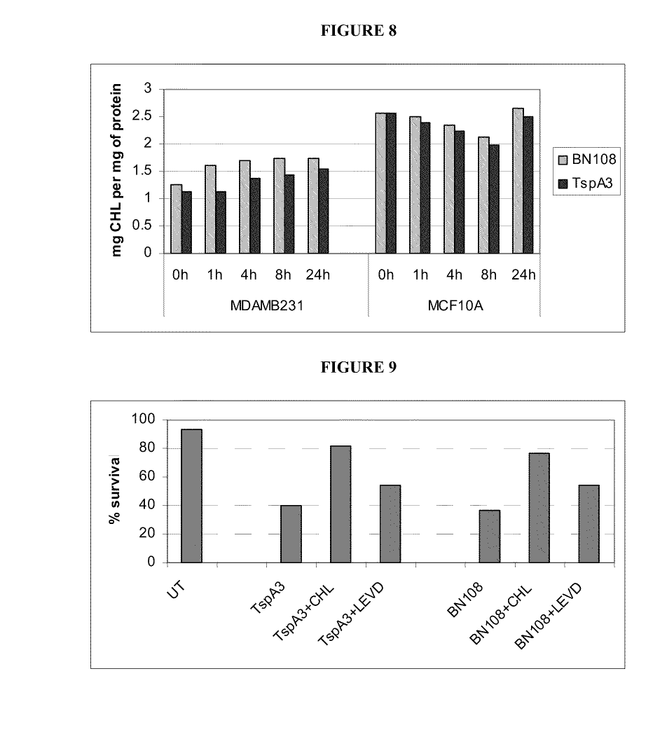

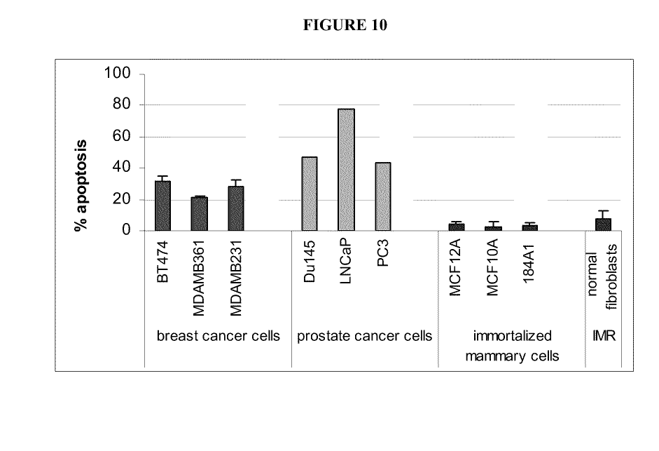

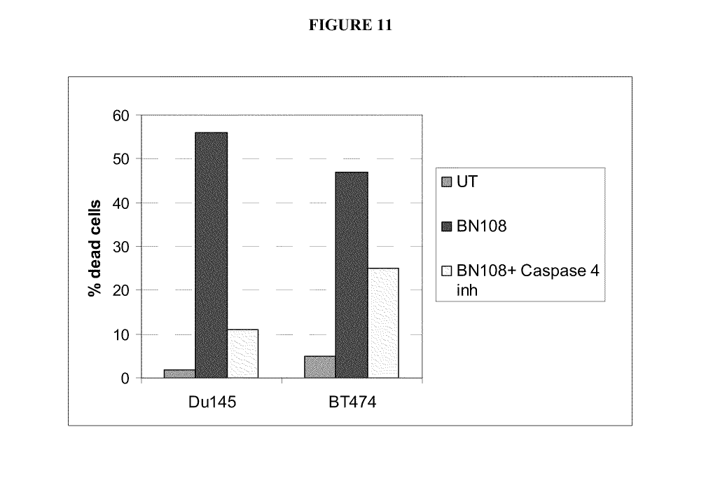

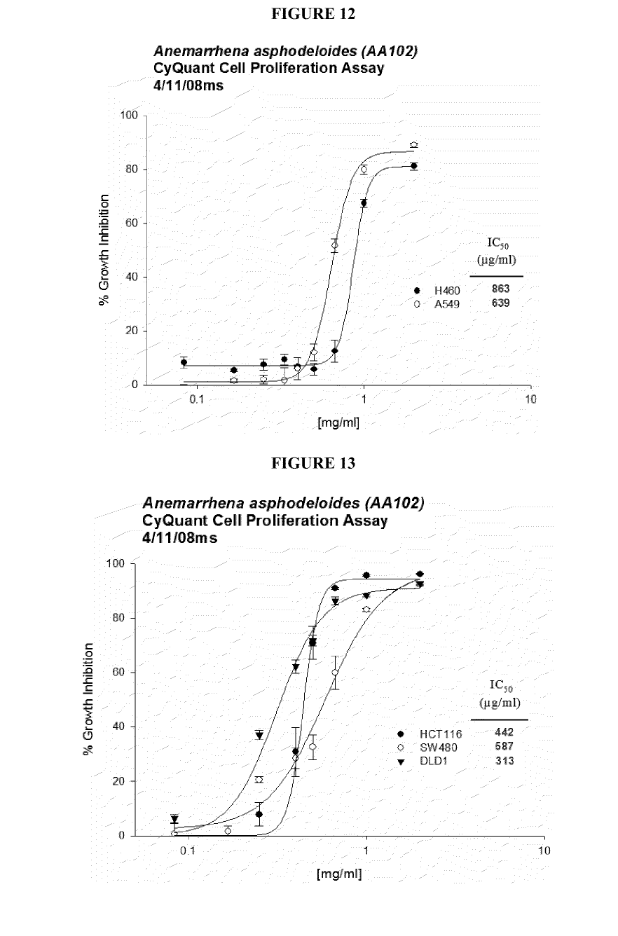

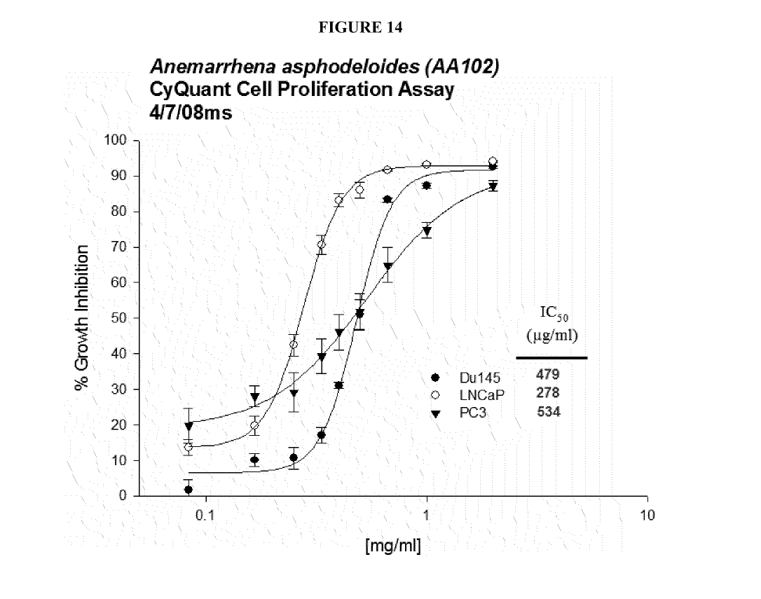

BN108 and Timosaponin A3 had similar effects on cholesterol (CHL) synthesis in cancerous and normal cells. As can be seen in FIG. 8, Timosaponin A3 and BN108 tended to increase cholesterol synthesis slightly in cancerous (MM23) breast cells and reduce cholesterol synthesis, at least through the first 8 hours after treatment, in normal (MCF10A) breast cells.

The cytotoxic effects of BN108 and Timosaponin A3 are attenuated by cholesterol (CHL) and the caspase 4 inhibitor LEVD. As can be seen in FIG. 9, untreated (UT) BT474 cells were over 90% viable 24 hours after treatment with control solution, but only 40% of cells treated with either Timosaponin A3 or BN108 survived 24 hours (based upon FACScan for permeability of dead cells to propidium iodide and binging of Annexin V). Treatment of BT474 cells with 250 mM cholesterol protected the cells from the effects of Timosaponin A3 and BN108, with about 80% of the cells surviving 24 hours in the presence of cholesterol (CHL). A similar effect was noted in the presence of the caspase 4 inhibitor LEVD. As can be seen in FIG. 9, treatment with LEVD resulted in an attenuation of the effects of Timosaponin A3 and BN108, with between 55 and 60% of the BT474 cells treated with LEVD, and either Timosaponin A3 or BN108, surviving for 24 hours post treatment. Results from this experiment are summarized in the table, below.

| | % early | % late | % Total | % Difference |
|---|---|---|---|---|
| Drug | apoptotic | apoptotic | apoptotic | from control |
| DMSO | 6 | 5 | 11 | — |
| BN108 | 50 | 15 | 65 | 54 |
| Timosaponin A3 | 35 | 33 | 68 | 57 |
| Timosaponin B2 | 7 | 5 | 12 | 1 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition for the treatment of cancer in a multicellular organism, comprising an amount of Timosaponin A3 and Timosaponin B2, which is effective to treat cancer in said multicellular organism.

2. The composition of claim 1, consisting of: an amount of Timosaponin A3 and Timosaponin B2 effective to treat cancer in said multicellular organism; and one or more members of the group consisting of excipients, binders, fillers, diluents, capsule formers, slow-release agents, flavorings and taste masking agents.

3. The composition of claim 1, containing a combined weight of about 50-1,000 mg of Timosaponin A3 and Timosaponin B2.

4. The composition of claim 1 in an oral dosage form.

5. A method for the treatment of cancer in a multicellular organism, comprising administering to said multicellular organism an effective amount of a pharmaceutical composition comprising Timosaponin A3 and Timosaponin B2.

6. The method of claim 5, wherein the pharmaceutical composition consists of: an amount of Timosaponin A3 and Timosaponin B2 effective to treat cancer in said multicellular organism; and one or more members of the group consisting of excipients, binders, fillers, diluents, capsule formers, slow-release agents, flavorings and taste masking agents.

7. A method for the treatment of cancer in a multicellular organism, comprising administering to said multicellular organism an effective amount of pharmaceutical composition comprising Timosaponin A3.

8. The method of claim 7, wherein the pharmaceutical composition consists essentially of an amount of Timosaponin A3 effective to treat cancer in said multicellular organism.

9. The method of claim 7, wherein the pharmaceutical composition consists of: an amount of Timosaponin A3 effective to treat cancer in said multicellular organism; and one or more members of the group consisting of excipients, binders, fillers, diluents, capsule formers, slow-release agents, flavorings and taste masking agents.

10. The method of claim 7, wherein the pharmaceutical composition contains about 50-1,000 mg of Timosaponin A3.

11. A method for the treatment of cancer in a multicellular organism, comprising administering to said multicellular organism an effective amount of pharmaceutical composition comprising Timosaponin B2.

12. The method of claim 11, wherein the pharmaceutical composition consists of: an amount of Timosaponin B2 effective to treat cancer in said multicellular organism; and one or more members of the group consisting of excipients, binders, fillers, diluents, capsule formers, slow-release agents, flavorings and taste masking agents.

13. The method of claim 11, wherein the pharmaceutical composition contains about 50-1,000 mg of Timosaponin B2.

14. The method of claim 11, wherein the pharmaceutical composition is in an oral dosage form.

15. The method of claim 11, wherein the multicellular organism is a rat, a mouse, a human, a simian or a dog.

16. The method of claim 15, wherein the multicellular organism is a human.

17. The method of claim 11, wherein the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

18. A method for selectively inducing apoptosis in a hyperproliferative population of cells in a multicellular organism, comprising administering to said multicellular organism an effective amount of a pharmaceutical composition comprising Timosaponin A3 and Timosaponin B2.

19. A method for selectively inducing apoptosis in a hyperproliferative population of cells in a multicellular organism, comprising administering to said multicellular organism an effective amount of pharmaceutical composition comprising Timosaponin A3.

20. The method of claim 19, wherein the pharmaceutical composition consists of: an amount of Timosaponin A3 effective to selectively induce apoptosis in a hyperproliferative population of cells in said multicellular organism; and one or more members of the group consisting of excipients, binders, fillers, diluents, capsule formers, slow-release agents, flavorings and taste masking agents.

21. A method for selectively inducing apoptosis in a hyperproliferative population of cells in a multicellular organism, comprising administering to said multicellular organism an effective amount of pharmaceutical composition comprising Timosaponin B2.

22. The method of claim 21, wherein the pharmaceutical composition consists of: an amount of Timosaponin B2 effective to selectively induce apoptosis in a hyperproliferative population of cells in said multicellular organism; and one or more members of the group consisting of excipients, binders, fillers, diluents, capsule formers, slow-release agents, flavorings and taste masking agents.

23. A composition for the treatment of cancer in a multicellular organism, comprising an amount of an extract of Anemarrhena asphodeloides Bunge, which is effective to treat cancer in said multicellular organism.

24. The composition of claim 23, consisting of: an amount of an extract of Anemarrhena asphodeloides Bunge effective to treat cancer in said multicellular organism; and one or more members of the group consisting of excipients, binders, fillers, diluents, capsule formers, slow-release agents, flavorings and taste masking agents.

25. The composition of claim 23, containing about 50-50,000 mg of an extract of Anemarrhena asphodeloides Bunge.

26. The composition of claim 23, wherein the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

27. A method for the treatment of cancer in a multicellular organism, comprising administering to said multicellular organism an effective amount of a pharmaceutical composition comprising an extract of Anemarrhena asphodeloides Bunge.

28. The method of claim 27, wherein the pharmaceutical composition consists of: an amount of an extract of Anemarrhena asphodeloides Bunge effective to treat cancer in said multicellular organism; and one or more members of the group consisting of excipients, binders, fillers, diluents, capsule formers, slow-release agents, flavorings and taste masking agents.

29. The method of claim 27, wherein the pharmaceutical composition contains about 50-50,000 mg of an extract of Anemarrhena asphodeloides Bunge.

30. The method of claim 27, wherein the multicellular organism is a human.

31. The method of claim 27, wherein the cancer is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.

32. A method for selectively inducing apoptosis in a hyperproliferative population of cells in a multicellular organism, comprising administering to said multicellular organism an effective amount of a pharmaceutical composition comprising an extract of Anemarrhena asphodeloides Bunge.

33. The method of claim 32, wherein the pharmaceutical composition consists of: an amount of an extract of Anemarrhena asphodeloides Bunge effective to selectively induce apoptosis in a hyperproliferative population of cells in said multicellular organism; and one or more members of the group consisting of excipients, binders, fillers, diluents, capsule formers, slow-release agents, flavorings and taste masking agents.

34. The method of claim 32, wherein the pharmaceutical composition contains a about 50-50,000 mg of an extract of Anemarrhena asphodeloides Bunge.

35. The method of claim 32, wherein the multicellular organism is a human.

36. The method of claim 32, wherein the hyperproliferative population of cells is selected from the group consisting of bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer and uterine cancer.
